(19) United States
(12) Patent Application Publication
(10) Pub. No.: US 2025/0347627 A1
(43) Pub. Date: Nov. 13, 2025

(54) APPARATUS FOR DETECTING RESPIRATORY VIRUSES IN REAL TIME

(71) Applicant: FIBERFOX, INC., Daegeon (KR)

(72) Inventors: Won Young KIM, Daejeon (KR); Sung Young PARK, Suwon-si (KR); Chang Ho KANG, Daejeon (KR)

(21) Appl. No.: 19/064,755

(22) Filed: Feb. 27, 2025

(30) Foreign Application Priority Data
May 10, 2024 (KR) 10-2024-0061561

(51) Int. Cl. G01N 21/67; G01N 1/24; G01N 33/497

(52) U.S. Cl. CPC G01N 21/67; G01N 1/24; G01N 33/497

(57) ABSTRACT

Proposed is a detection apparatus. The apparatus may include a detector configured to detect a virus in a specimen containing an exhaled breath of a user. A collection container may be provided to receive the specimen. The detector may be installed in the collection container and may detect a virus in the specimen entered into the collection container. The detector may detect relaxation energy of the specimen decomposed by at least one of thermal energy, light energy, and electrical energy, and the detector may detect the virus using a wavelength, an intensity value, or a ratio between intensity values by wavelength of the detected relaxation energy.

### CROSS REFERENCE TO RELATED APPLICATION

The present application claims priority to Korean Patent Application No. 10-2024-0061561, filed May 10, 2024, the entire contents of which is incorporated herein for all purposes by this reference.

### BACKGROUND

### Technical Field

The present disclosure relates to an apparatus for detecting respiratory viruses.

### Description of the Related Art

Several new respiratory infectious viruses, such as Ebola virus, SARS, MERS, and COVID-19, that cause disease in human beings have emerged in recent years and circulate widely.

These infectious diseases are associated with fever, and people with high fever may be initially considered as suspected cases of the disease. After a preliminary assessment, a detailed examination can be conducted to determine whether or not one is infected. To diagnose infection, samples are collected and self-test kits are used. However, PCR/antigen self-test kits have many problems, such as taking a long time (from 30 minutes to 24 hours) to produce results, the sample collection through nasopharyngeal swab can be uncomfortable for a patient, and the kit must be disposed of after the test.

### SUMMARY

Accordingly, the present disclosure has been made keeping in mind the above problems occurring in the related art, and the present disclosure is intended to provide a detection apparatus that can detect viruses contained in the breath exhaled by a user in real time.

In order to achieve the above objective, according to an embodiment of the present disclosure, there is provided a detection apparatus.

The apparatus may include a detector configured to detect a virus in a specimen containing an exhaled breath of a user.

A collection container may be provided to receive the specimen.

The detector may be installed in the collection container and may detect a virus in the specimen entered into the collection container.

When a force of a user's exhalation is defined as a first force, a cover that receives the specimen moving by the first force may be provided at an entrance of the collection container.

An input flow path connected to the detector may be formed in the center of the cover.

The specimen entered into the cover may move along the input flow path and may be delivered to the detector.

When the specimen is input into the collection container along a first direction, a cover installed at an entrance of the collection container may be provided.

A guide hole may be provided in the center of the cover.

The guide hole may have an inner diameter that decreases along the first direction.

An inspection box that is installed inside the collection container and has a receiving space for accommodating the specimen may be provided.

The inspection box may be connected directly to an end of the guide hole, or may be connected to the end of the guide hole through a guide pipe.

The detector may detect a virus in the specimen contained in the inspection box.

A foreign substance filter may be provided at the end of the guide hole to be detachable and to filter out foreign substances.

Non-woven fabric for masks may be used for the exterior and lining of the foreign substance filter.

Polypropylene nonwoven filter fabric or a dust collection filter may be used for the body of the foreign matter filter disposed between the exterior and the lining.

As the dust collection filter, a cellulose filter, a polypropylene (PP) filter, and an electric type (a method of ionizing inhaled pollutant particles and attaching the ionized particles to a dust collection cell by electrostatic force) filter may be used. Alternatively, as the dust collection filter, a filter made of stainless steel, a steel alloy containing chromium, or aluminum, a photocatalytic zeolite filter, etc. may be used.

Meanwhile, an exhalation sensor may be provided on one side of the front of the filter (the side facing the user). The exhalation sensor may sense when the user exhales. The detection apparatus may initiate operation when the user's exhalation is detected through the exhalation sensor. That is, when exhaled breath is recognized by the exhalation sensor, the detection apparatus may generate a spark necessary for detecting a virus. To initiate operation immediately in response to the exhalation sensor, the detection apparatus may maintain a standby state in which the power is turned on to obtain the driving power necessary for spark generation.

A suction fan may be installed in the collection container and arranged to face the foreign substance filter with the inspection box in between.

The suction fan may be provided to suck gas in the receiving space.

When the user's exhaled breath moves along a first direction, the collection container may be provided to extend along the first direction.

An inspection box may be provided on a first side in the collection container in the first direction.

A receiving space in which the specimen is received may be formed in the inspection box.

The detector may detect a virus in the specimen contained in the inspection box.

A driving unit for driving the detector may be provided on a second side in the collection container.

An exhaust flow path extending along a second direction inclined to the first direction may be formed in the center of the collection container in the first direction.

An output hole for the exhaust flow path may be formed on a bottom of the collection container.

The exhaust flow path may be connected to an output end of the inspection box.

The detector may detect relaxation energy of the specimen decomposed by at least one of thermal energy, light energy, and electrical energy.

The detector may detect the virus using a wavelength or an intensity value of the detected relaxation energy, or may detect the virus using a ratio between intensity values by wavelength of the detected relaxation energy of multiple wavelengths.

An inspection box may be provided with a receiving space for accommodating the specimen.

A collector may be provided that collects the specimen introduced into the receiving space to a set position in the receiving space.

An optical device may be provided that focuses on the set position.

The detector may detect the virus through analysis of energy passing through the optical device.

An inspection box may be provided with a receiving space for accommodating the specimen.

A discharge electrode may be provided at a first position in the receiving space.

A ground electrode may be provided at a second position in the receiving space.

A power device may be provided to apply a high voltage between the discharge electrode and the ground electrode.

An optical device may be provided that focuses on a set position closer to the second position than the first position on a virtual straight line connecting the first position and the second position.

The detector may detect the virus through analysis of energy passing through the optical device.

A detection electrode may be provided at the set position,

The detection electrode may create a spark that produces plasma targeting the specimen.

The detector may detect relaxation energy of the specimen decomposed by the spark.

The detector may detect the virus using a wavelength of the detected relaxation energy.

An inspection box may be provided with a receiving space for accommodating the specimen.

A sensor may be provided to detect relaxation energy in the receiving space by wavelength.

The detector may detect the virus by using a wavelength and an intensity value at which the relaxation energy is detected, or may detect the virus by using a ratio between intensity values by wavelength of the detected relaxation energy of multiple wavelengths.

An inspection box may be provided with a receiving space for accommodating the specimen.

The inspection box may be formed in a polyhedral shape.

A first optical device installed on a first surface of a plurality of surfaces constituting the inspection box may be provided.

A second optical device installed on a second surface that is distinct from the first surface among the plurality of surfaces constituting the inspection box may be provided.

When a normal line of the first surface is defined as a first normal line and a normal line of the second surface is defined as a second normal line, the first surface and the second surface may be formed so that the first normal line and the second normal line intersect at a set position.

The first surface and the second surface may have the same distance to the set position.

The first optical device may be provided with a first detection filter that passes only energy of a first wavelength.

The second optical device may be provided with a second detection filter that passes only energy of the second wavelength.

A first sensor may be provided to sense energy that has passed through the first detection filter.

A second sensor may be provided to sense energy passing through the second detection filter.

The detector may detect the virus using an output value of the first sensor and an output value of the second sensor.

An inspection box may be provided with a receiving space for accommodating the specimen.

A detection electrode may be provided in the receiving space.

The detection electrode may create a spark that produces plasma targeting the specimen.

The detector may detect relaxation energy of the specimen decomposed by the spark.

The detector may detect the virus using a wavelength or an intensity value of the detected relaxation energy, or may detect the virus using a ratio between intensity values by wavelength of the detected relaxation energy of multiple wavelengths.

A sterilizer may be provided to disinfect the receiving space when a virus detection process of the detector targeting a specific user is completed.

The sterilizer may output disinfected water containing an oxygen compound toward the detection electrode.

The detection electrode may electrolyze the disinfected water to generate oxygen.

The receiving space may be disinfected by the oxygen generated in the receiving space.

An inspection box may be provided with a receiving space for accommodating the specimen.

A detection electrode may be provided in the receiving space,

The detection electrode may create a spark that produces plasma targeting the specimen.

An optical tube connected to the inspection box may be provided.

The optical tube may be equipped with a lens, a multi-filter, and a multi-sensor.

The lens may create an optical path through which inspection light corresponding to relaxation energy formed by the plasma propagates.

The multi-filter may divide the inspection light by wavelength so that when the inspection light is input, the inspection light may be output toward different locations for each wavelength.

The multi-sensor may receive the inspection light output from the multi-filter at different locations (relative to the optical tube, the multi-filter, or the lens).

The multi-sensor may output information about a location where the inspection light is received.

The detector may extract a combination of information about a location where the inspection light is received from a lookup table obtained through experiment.

The detector may output information on a virus matching the extracted combination.

According to the present disclosure, real-time detection of respiratory viruses using plasma can dramatically reduce testing time.

According to the present disclosure, blowing into the detection apparatus of the present disclosure can provide an environment where viruses can be detected in real time.

This can minimize user inconvenience related to virus testing. In addition, since rapid testing is possible, the detection apparatus can be applied to environments where viruses need be tested or detected for multiple users.

The detection apparatus of the present disclosure can detect viruses contained in the breath exhaled by a user by using arc plasma or arc discharge.

Viruses that respond to arc plasma or arc discharge may produce energy of a unique wavelength, such as light energy.

The present disclosure can provide a detection apparatus that, by using a detection filter that matches the unique wavelength emitted by a specific virus, receives only light energy of that specific wavelength.

In other words, the detection apparatus can detect the presence of a specific virus based on whether or not light of a specific wavelength is received.

The detection apparatus of the present disclosure provides an inspection box in which a plurality of detection filters can be installed. The inspection box can be formed into a polyhedral structure. An optical tube having a detection filter can be installed on each face forming the polyhedron.

According to the inspection box, the focus of the optical devices, for example, lenses, provided in all the optical tubes can be equally aligned to the center of the inspection box. Accordingly, a detection electrode that generates the arc plasma or arc discharge can be positioned at the center of the inspection box.

When a user exhales along a first direction, a suction fan may be provided at the back of the inspection box to suck in air inside the inspection box along the first direction. Due to the suction fan, the breath exhaled by the user can be sucked into the inspection box. In addition, since the air inside the inspection box moves to the suction fan, the air in the inspection box can be prevented from being discharged toward the next user.

An exhaust flow path can be formed that induces air drawn in through the suction fan to be discharged in a downward direction inclined in the first direction. The exhaust flow path directs the user's breath towards the ground, which can prevent other users from inhaling air containing the breath of the previous user.

In addition, according to the present disclosure, a sterilizer may be provided that supplies disinfected water toward the detection electrode.

Disinfected water dropped or sprayed onto the detection electrode by the sterilizer can be electrolyzed by the arc discharge of the detection electrode. Disinfectant gas is generated through electrolysis, and the interior space of the inspection box can be disinfected by the disinfectant gas. Although, according to the present embodiment, the detection electrode also has the function of generating disinfectant gas, a separate disinfectant electrode that performs the function of generating disinfectant gas may be provided.

Due to the disinfected inspection box, the situation where the next user's virus test is affected by the exhaled breath of the previous user can be prevented.

In addition, in the present disclosure, a means may be provided to attract or capture the user's breath entering the inspection box to the detection electrode.

For example, a discharge electrode and a ground electrode that react with each other may be provided in the inspection box. When a high voltage is applied to the discharge electrode, unipolar air ions may be generated at the end of the discharge electrode by corona charging. The generated air ions may move toward the ground electrode by the electric field. A specimen containing the user's breath that enters the inspection box becomes charged while colliding with the air ions moving toward the ground electrode. The charged specimen may move to the ground electrode. In addition, the ground electrode may be formed on the bottom of the inspection box so as to be affected by gravity. Due to this, the speed or amount of air ions toward the ground electrode is increased, which can help increase the amount of specimen toward the ground electrode.

The detection electrode may be formed directly above the ground electrode. Due to this, the detection electrode can operate in an area or location where the amount of specimen required for virus detection is secured. Therefore, the detection apparatus of the present disclosure can provide an environment in which a virus can be detected using only the user's exhaled breath.

The detection apparatus of the present disclosure may adopt a wavelength detection system that detects only the presence or absence of energy of a specific wavelength that has passed through a filter, and detect the presence or absence of a virus using binarized data by wavelength. The detection apparatus can easily increase the number of types of viruses that can be measured at one time by increasing the number of wavelength detection systems. Accordingly, various methods for increasing wavelength detection systems can be provided.

According to the present disclosure, infectious diseases can be prevented by detecting respiratory viruses, such as COVID-19, COVID-19 variants, rhinovirus, and influenza virus, as well as foot-and-mouth disease virus and avian influenza. The present disclosure can also be applied to alcohol tests, detection of biochemical anthrax, smallpox, and polio viruses, environmental monitoring, water quality management, etc.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a schematic view showing a detection apparatus according to an embodiment of the present disclosure;

FIG. 2 is a schematic view showing the inside of a detection apparatus according to an embodiment of the present disclosure;

FIG. 3 is a cross-sectional view showing the inside of a detection apparatus according to an embodiment of the present disclosure;

FIG. 4 is a schematic view showing the operation of a collector according to an embodiment of the present disclosure;

FIG. 5 is a schematic view showing an inspection box according to an embodiment of the present disclosure;

FIG. 6 is a schematic view showing a detector according to an embodiment of the present disclosure;

FIG. 7 is a schematic view showing a detector according to another embodiment of the present disclosure;

FIG. 8 is a schematic view showing a detector according to still another embodiment of the present disclosure; and

FIG. 9 is a block view showing a detector according to an embodiment of the present disclosure.

FIG. 10 is a diagram showing a computing device according to an embodiment of the present disclosure.

### DETAILED DESCRIPTION

Hereinafter, with reference to the accompanying drawings, embodiments of the present disclosure will be described in detail so that those skilled in the art may easily carry out the present disclosure. The present disclosure may be embodied in many different forms and is not limited to the embodiments set forth herein. In order to clearly describe the present disclosure, parts irrelevant to the description are omitted in the drawings, and same reference numerals are assigned to the same or similar components throughout the specification.

In this specification, redundant descriptions of the same components are omitted.

In addition, in this specification, when a component is said to be “connected (or coupled)” to another component, this includes not only the case of being “directly connected (or coupled)” but also “indirectly connected (or coupled)” with another component in between. On the other hand, when a component is said to be “directly connected (or coupled)” to another component, it should be understood that there are no other components in between.

The terms used in this specification are only used to describe specific embodiments, and are not intended to limit the present disclosure.

The singular expression may include a plural expression unless the context clearly indicates otherwise.

In addition, it should be understood that in the present disclosure, terms such as “comprise” and “have” are intended to designate that a feature, number, step, operation, component, part, or combination thereof described in the specification exists, and do not preclude the possibility of addition or existence of one or more other features or numbers, steps, operations, components, parts, or combinations thereof.

As used herein, the term “and/or” includes a combination of a plurality of listed items or any of a plurality of listed items. As used herein, “A or B” may include “A”, “B”, or “both A and B”.

In this specification, detailed descriptions of well-known functions and configurations that may obscure the gist of the present disclosure will be omitted.

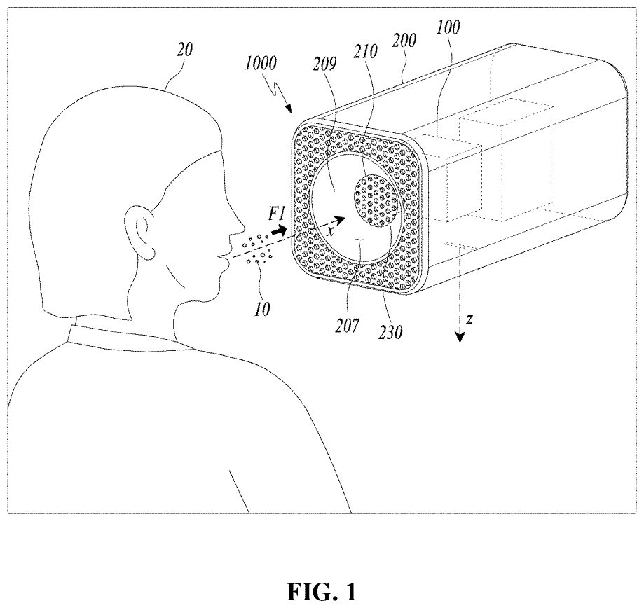

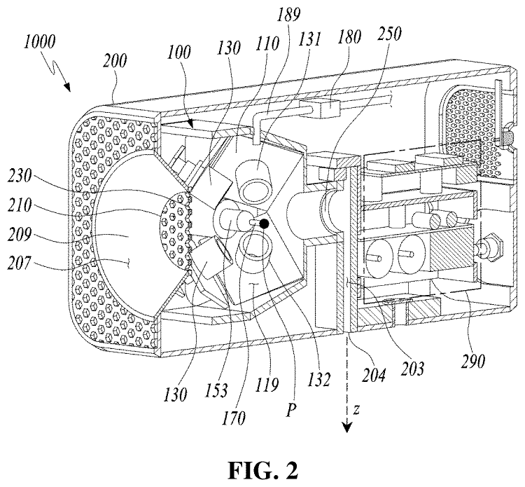

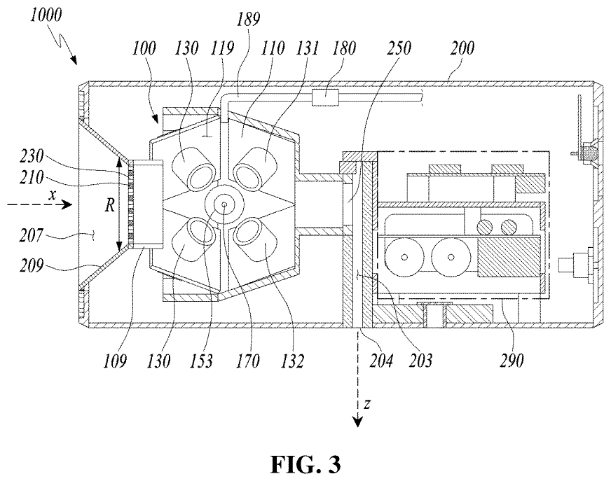

FIG. 1 is a schematic view showing a detection apparatus 1000 according to an embodiment of the present disclosure. FIG. 2 is a schematic view showing the inside of the detection apparatus 1000 according to an embodiment of the present disclosure. FIG. 3 is a cross-sectional view showing the inside of the detection apparatus 1000 according to an embodiment of the present disclosure.

The detection apparatus 1000 illustrated in the drawings may include a detector 100.

The detector 100 may detect a virus in a specimen 10 contained in the exhaled breath of a user 20.

Analyzing the specimen 10 scattered in the air may be difficult because the density of the specimen 10 propagated in the air decreases rapidly with the distance traveled.

In order to prevent the density reduction of the specimen 10 to the maximum extent, a collection container 200 for receiving the specimen 10 may be provided in the detection apparatus 1000. The collection container 200 may be formed in a barrel shape for collecting the specimen 10 scattered in the air into one place. Air that flows into the barrel-shaped collection container 200 may be blocked by the collection container 200 and may not be discharged to the outside, but may be collected inside the collection container 200.

The detector 100 may be installed in the collection container 200. Then, the detector 100 may detect a virus in the specimen 10 input into the collection container 200 and collected inside the collection container 200.

When the force of exhalation by the user 20 is defined as a first force F1, a cover 210 that receives the specimen 10 moving by the first force FI may be provided at the entrance of the collection container 200. The cover 210 may be formed with a structure for collecting the maximum amount of the specimen 10.

The specimen 10 may be moved along a first direction x by the first force F1 and input into the collection container 200. At this time, the collection container 200 may be placed on an imaginary line extending along the first direction x. From another perspective, this may correspond to a situation where the user 20 places his/her mouth against the entrance of the collection container 200 and blows air into the collection container 200 along the length direction of the collection container 200.

An input flow path 209 connected to the detector 100 may be provided in the center of the cover 210. The specimen 10 input into the cover 210 may move along the input flow path 209 and be delivered to the detector 100.

A guide hole 207 may be provided in the center of the cover 210. The guide hole 207 may form the input flow path 209 through which the specimen 10 flows. In addition, the guide hole 207 may be formed in a structure in which the maximum amount of the specimen 10 exhaled by the user 20 may be introduced.

As an example, the guide hole 207 may be formed larger than the size of the mouth of the user 20 when opened to its maximum. To be specific, an inner diameter R of the entrance of the guide hole 207 may be 10 to 20 cm. To increase the density of the specimen 10 introduced through the guide hole 207, the guide hole 207 may be formed in a shape in which the inner diameter R decreases along the first direction x, i.e., in a so-called funnel shape.

An inspection box 110 may be installed inside the collection container 200. A receiving space 119 for receiving the specimen 10 may be formed in the inspection box 110.

The inspection box 110 may be directly connected to the end of the guide hole 207 with the minimum inner diameter R, or may be connected to the end of the guide hole 207 through a guide pipe 109.

The detector 100 may detect a virus in the specimen 10 accommodated in the receiving space 119 of the inspection box 110.

A foreign substance filter 230 may be provided to prevent various foreign substances from entering the detector 100. The foreign substance filter 230 may be formed detachably at the end of the guide hole 207. The foreign substance filter 230 may have as a mesh structure that allows the specimen 10 to pass through and filters out foreign substances.

Meanwhile, the foreign substance filter 230 functions as a resistance that impedes the flow of the specimen 10, and thus may impede the specimen 10 from flowing into the inspection box 110. A means for drawing the specimen 10 toward the inspection box 110 may be provided so that the specimen 10 overcomes the impediment of the foreign substance filter 230 and flows into the inspection box 110.

For example, a suction fan 250 may be installed in the collection container 200 and positioned facing the foreign substance filter 230 with the inspection box 110 in between.

The suction fan 250 may be configured to suck gas from the receiving space 119.

For example, assuming that the exhaled breath of the user 20 moves along the first direction x, the collection container 200 may be formed to extend along the first direction x. At this time, the suction fan 250 may be positioned downstream of the cover 210 and the inspection box 110 in the first direction x, and may rotate in a direction to suck in air contained in the inspection box 110. From another perspective, the suction fan 250 may include a propeller or blade that causes a flow of fluid to flow along the first direction x when rotating in the forward direction.

In the first direction x, the inspection box 110 may be provided on one side in the collection container 200.

A driving unit 290 for driving the detector 100 may be provided on the other side in the collection container 200.

In the first direction, an exhaust flow path 203 extending along a second direction z inclined to the first direction x may be formed in the center of the collection container 200. As an example, the second direction z may include a direction perpendicular to the first direction x. The exhaust flow path 203 may be formed between the inspection box 110 and the driving unit 290.

An output hole 204 of the exhaust flow path 203 may be formed on the bottom surface of the collection container 200.

The exhaust flow path 203 may be connected to the output end of the inspection box 110. In the present embodiment, air contained inside the inspection box 110 may move along the exhaust flow path 203 and be discharged to the outside through the output hole 204 of the exhaust flow path 203 formed on the bottom surface of the collection container 200. The air discharged from the bottom of the collection container 200 may be discharged toward the ground. The air may contain a virus. Thus, when the virus is released toward the next user 20, an examinee may become infected with the virus during the examination process. According to the present embodiment, since the air is discharged toward the ground and not toward the next user 20 can be prevented from being infected with the virus during the examination process.

The detector 100 may detect relaxation energy of the specimen 10 decomposed by at least one of thermal energy, light energy, and electrical energy.

The detector 100 may detect a virus using the wavelength or intensity value of the detected relaxation energy. Alternatively, the detector 100 may detect a virus by using the ratio between intensity values of the detected individual relaxation energies of multiple wavelengths. In other words, the detector 100 may detect a virus by using the ratio between intensity values for each wavelength of the detected relaxation energy.

Below, the explanation will focus on the case where the relaxation energy is light energy.

In order to detect relaxation energy, it is necessary to densely pack the specimen 10 in the location where the inspection is actually performed within the inspection box 110.

To ensure the reliability of virus detection, the larger the amount of specimen 10, the more advantageous it is. However, if the same user 20 is made to exhale deeply multiple times to obtain a large amount of specimen 10, discomfort may occur to the user 20. Thus, it may be desirable to detect a virus using roughly exhaled breath considering the convenience of the user 20. In this case, however, the amount of specimen 10 required for virus detection may be unsatisfactory. In order to solve this problem, the cover 210 having the guide hole 207 may be used as a means to gather the breath exhaled by the user 20 into one place as much as possible. A collector 150 that collects the specimen 10 inside the inspection box 110 into one place may be additionally provided.

The collector 150 may capture the specimen 10 introduced into the receiving space 119 to a set position p of the receiving space 119.

An optical device 130 focusing on the set position p may be provided. For example, the optical device 130 may include a lens 120 or a mirror focusing on the set position p. The detector 100 may detect a virus by analyzing light energy passing through the optical device 130.

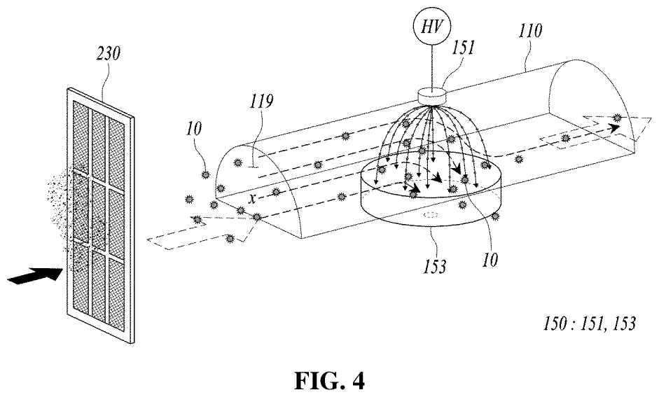

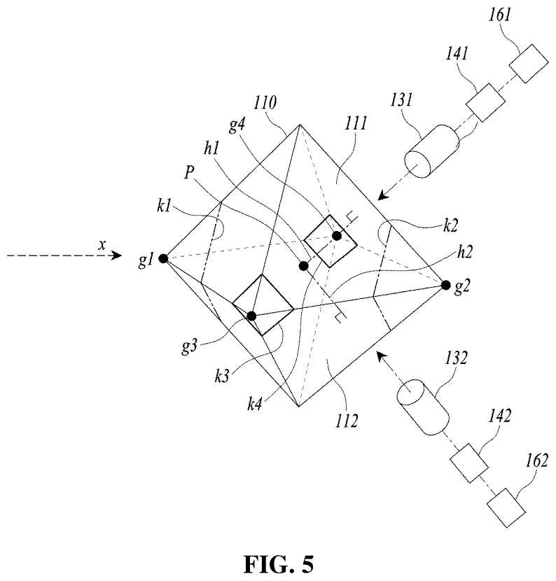

FIG. 4 is a schematic view showing the operation of the collector 150 according to an embodiment of the present disclosure. FIG. 5 is a schematic view showing the inspection box 110 according to an embodiment of the present disclosure.

The detection apparatus 1000 may be provided with a discharge electrode 151 formed in a first position of the receiving space 119 of the inspection box 110. The detection apparatus 1000 may also be provided with a ground electrode 153 formed in a second position of the receiving space 119 of the inspection box 110. In addition, a power device that applies a high voltage between the discharge electrode 151 and the ground electrode 153 may be provided. As an example, the power device may be included in the driving unit 290 provided inside the collection container 200.

When a high voltage HV is applied to the discharge electrode 151, unipolar air ions may be generated at the end of the discharge electrode 151 by corona charging. The generated air ions may move toward the ground electrode 153 by the electric field or an electric force line ef. The specimen 10 introduced into the inspection box 110 becomes charged when colliding with the air ions moving toward the ground electrode 153. The charged specimen 10 may move to the ground electrode 153. In addition, the ground electrode 153 may be formed on the bottom of the inspection box 110 so as to be affected by gravity. Due to this, the speed or amount of air ions moving toward the ground electrode 153 is increased, which can help increase the amount of specimen 10 toward the ground electrode 153.

Assuming an imaginary straight line connecting the first position and the second position, it is preferable that the imaginary straight line extend along a direction inclined to the first direction in which the specimen 10 moves.

For example, when the specimen 10 moves along a horizontal direction parallel to the ground, the first position where the discharge electrode 151 is placed may be set above the movement path of the specimen 10. The second position where the ground electrode 153 is placed may be set below the movement path of the specimen 10.

The optical device 130 for photographing the specimen 10 may have the set position p, which is closer to the second position than the first position on the imaginary straight line connecting the first position and the second position, as a focus. Preferably, the set position p is created between the first position and the second position, and may be created closer to the second position than to the first position. According to the present embodiment, the specimen 10 moving toward the ground electrode 153 by the discharge electrode 151 may pass through the detection electrode 170 provided at the set position p during the process of moving toward the ground electrode 153. To summarize, due to the discharge electrode 151 and the ground electrode 153, the specimen 10 introduced into the inspection box 110 is captured toward the detection electrode 170, and in this process, the density of the specimen 10 at the location of the detection electrode 170 may be significantly increased. The specimen 10 with increased density may satisfy the density condition required to detect a virus.

In the drawing, the end of the detection electrode 170 is placed at the set position p. However, alternatively, the end of the detection electrode 170 may be positioned so that the relaxation energy associated with the largest amount of the specimen 10 is generated at the set position p. The position of the end of the detection electrode 170 at this time may be slightly spaced from the set position p, and even in this case, the detection electrode 170 may be treated as being provided at the set position p in this specification.

The detection electrode 170 provided in the receiving space 119 may create a spark that produces plasma targeting the specimen 10. The electric energy required to generate the plasma may be supplied to the detection electrode 170 by the driving unit 290.

The detector 100 may detect relaxation energy of the specimen 10 decomposed by a spark.

The detector 100 may detect a virus by using the wavelength and intensity value of the detected relaxation energy. Alternatively, the detector 100 may detect a virus by using the ratio of intensity values by wavelength of the detected relaxation energy of multiple wavelengths.

The detector 100 may detect a virus by analyzing energy, for example, light energy, that passes through the optical device 130. As an example, the optical device 130 may include the lens 120 or mirror that receives or collects light energy at the set position p.

The detector 100 may be provided with a sensor 160 that detects the relaxation energy in the receiving space 119 by wavelength.

The detector 100 may detect a virus by using the wavelength and intensity value at which the relaxation energy is detected. Alternatively, the detector 100 may detect a virus by using the ratio between intensity values by wavelength of the detected relaxation energy of multiple wavelengths.

A method may be developed to clearly distinguish relaxation energy by wavelength.

It is assumed that the relaxation energy is light energy, and an image sensor is provided to convert the received light energy into an electrical signal.

The inspection box 110 may be formed in a polyhedral shape. In FIG. 5, an octahedral-shaped inspection box 110 is shown.

The optical device 130 may be installed on each side of the inspection box 110. A flow path for the specimen 10 or a variety of electrodes may be provided at the vertex of the inspection box 110.

In the octahedral inspection box 110 shown in FIG. 5, a first area k1 including a first vertex g1 may be connected to the guide hole 207 of the cover 210. Accordingly, the first area k1 may function as an entrance through which the specimen 10 is input.

A second area k2 including a second vertex g2 located on the opposite side of the first vertex g1 may be connected to the exhaust flow path 203. Accordingly, the second area k2 may function as an outlet through which the specimen 10 is output. The first area k1 and the second area k2 may be arranged on a straight line extending along the first direction x.

One selected among a plurality of vertices arranged between the first vertex g1 and the second vertex g2 may be defined as a third vertex g3. The discharge electrode 151 constituting the collector 150 may be installed in a third area k3 including the third vertex g3. The ground electrode 153 may be installed in a fourth area k4 including a fourth vertex g4 located opposite the third vertex g3.

The ground electrode 153 may be formed to protrude from the inner surface of the inspection box 110 toward the center of the receiving space 119. The ground electrode 153 may be formed in a ring shape. A protrusion made of an insulating material may be formed to protrude in the center of the ground electrode 153, and the detection electrode 170 may be formed in the center of the protrusion.

When referring to the positions of the third vertex g3 and the fourth vertex g4 shown in FIG. 5, the discharge electrode 151 and the ground electrode 153 may be arranged along the horizontal direction parallel to the ground rather than the direction of gravity. When a sterilizer 180 is provided to disinfect the inspection box 110 using disinfected water, the sterilizer 180 may be installed in the upper vertex area of the inspection box 110 to allow the disinfected water to freely fall. To avoid physical interference with the sterilizer 180, the discharge electrode 151 and the ground electrode 153 may be arranged along the horizontal direction.

A first optical device 131 installed on a first surface 111 of the plurality of surfaces forming the inspection box 110 may be provided.

A second optical device 132 installed on a second surface 112 that is distinct from the first surface 111 among the plurality of surfaces forming the inspection box 110 may be provided.

The normal line of the first surface 111 may be defined as a first normal line h1, and the normal line of the second surface 112 may be defined as a second normal line h2.

The first surface 111 and the second surface 112 may be provided such that the first normal line h1 and the second normal line h2 intersect at the set position p. The first surface 111 and the second surface 112 may have the same distance to the set position p. Due to this, each of the optical devices 130 provided on each side may use the same set position p as a common focus. This may mean that the plurality of optical devices 130 may all image the same specimen 10.

When utilizing an environment in which the plurality of optical devices 130 image the same specimen 10, the concept of distributing imageable wavelengths to the individual optical devices 130 may be applied.

As an example, the first optical device 131 may be provided with a first detection filter 141 that passes only energy of a first wavelength.

The second optical device 132 may be provided with a second detection filter 142 that passes only energy of a second wavelength.

Accordingly, a first sensor 161 that senses energy that has passed through the first detection filter 141 may be provided. A second sensor 162 may be provided to sense energy passing through the second detection filter 142.

The detector 100 may detect a virus using the output value of the first sensor 161 and the output value of the second sensor 162.

For example, the output value of each sensor 160 may be binarized. In other words, the output value of each sensor 160 may be either “0” or “1”.

When light energy of the first wavelength exists, the first sensor 161 may output 1. When light energy of the first wavelength does not exist, the first sensor 161 may output 0.

When light energy of the second wavelength exists, the second sensor 162 may output 1. When light energy of the second wavelength does not exist, the second sensor 162 may output 0.

Through preliminary experiments, it can be determined that a first virus has light energy of the first wavelength and does not have light energy of the second wavelength. When the output value of the first sensor 161 indicating the corresponding situation is 1 and the output value of the second sensor 162 is 0, the detector 100 may determine that the specimen 10 introduced into the inspection box 110 contains the first virus.

The sterilizer 180 may be provided to disinfect the receiving space 119 when the virus detection process of the detector 100 targeting a specific user 20 is completed.

The end of a disinfection pipe 189 through which disinfected water is output may be provided on the detection electrode in the direction of gravity. The disinfection pipe 189 is connected to the sterilizer 180, and the sterilizer 180 may be provided with a valve to open and close the disinfection pipe. The other end of the disinfection pipe may be connected to a disinfected water storage tank inside or outside the collection container 200.

The sterilizer 180 may output disinfected water containing an oxygen compound toward the detection electrode 170. As an example, disinfected water may contain hydrogen peroxide (H₂O₂).

The detection electrode 170 may electrolyze the disinfected water to generate oxygen.

The receiving space 119 may be disinfected by the oxygen generated in the receiving space 119. Due to the disinfected receiving space 119, virus detection of the next user 20 may be performed normally.

In this embodiment, the disinfected water is electrolyzed using the detection electrode 170 that excites the specimen 10 again. Alternatively, a separate disinfection electrode that electrolyzes the disinfected water may be provided in the inspection box 110. Additionally, the virus detection device may be equipped with a UV sterilizer. The UV sterilizer may sterilize exhaled breath (air) discharged to the outside of the collection container. At this time, the UV wavelength of 222 nm or 275 nm may be used, but is not limited thereto.

The detector 100 may be provided with an optical tube 139, the optical device 130, a multi-filter, and the sensor 160.

The optical device 130 shown in the drawing may include the lens 120.

The optical tube 139 is installed on each side of the inspection box 110, and each optical tube 139 may be provided with the lens 120, a detection filter 140, and the sensor 160.

According to this embodiment, a plurality of optical tubes 139 including the lens 120, the detection filter 140, and the sensor 160 may be provided. However, the plurality of optical tubes 139 may make it difficult to miniaturize the collection container 200.

In order to miniaturize the collection container 200, a method may be prepared to integrate the plurality of optical tubes 139 into one.

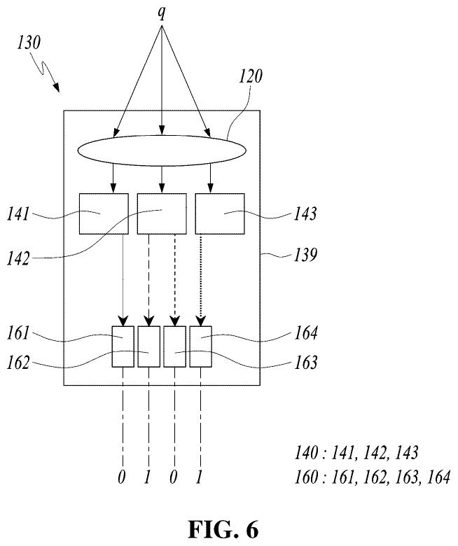

FIG. 6 is a schematic view showing the detector 100 according to an embodiment of the present disclosure.

A single optical tube 139 is connected to the inspection box 110, and the lens 120, a multi-filter, and a multi-sensor may be provided in the optical tube 139.

The lens 120 may create an optical path through which inspection light corresponding to relaxation energy q created by plasma propagates.

The multi-filter may divide inspection light by wavelength so that when inspection light is input, the inspection light is output toward different locations for each wavelength.

The multi-filter of FIG. 6 may include a plurality of detection filters 140 arranged within the optical tube 139. At this time, each detection filter 140 may be provided to pass only relaxation energy of a specific wavelength.

As shown in FIG. 6, the first detection filter 141 that outputs only the relaxation energy of the first wavelength when relaxation energy is input may be provided. The second detection filter 142 may be placed next to the first detection filter 141. The second detection filter 142 may output relaxation energy of the second wavelength from one side and output relaxation energy of a third wavelength from the other side when relaxation energy is input. A third detection filter 143 may be placed next to the second detection filter 142. The third detection filter 143 may output relaxation energy of a fourth wavelength when relaxation energy is input.

The multi-sensor may receive the inspection light output from the multi-filter at different locations. The multi-sensor may output information about the location where the inspection light is received.

The detector 100 may extract a combination of information about the location where the inspection light was received from a lookup table obtained through experiment.

The detector 100 may output information on a virus matching the extracted combination.

The multi-sensor in FIG. 6 may include a plurality of sensors 160 arranged within the optical tube 139. At this time, each sensor 160 may output the detection result of relaxation energy as a value corresponding to “1” or “0”.

The placement position of each sensor 160 is predefined and may represent the number of digits of the so-called measured value.

In the drawing, the first sensor 161 on the far left receives relaxation energy of the first wavelength and may represent the first left digit of the 4-digit measurement value. The second sensor 162, second from the left, receives relaxation energy of the second wavelength and may represent the second digit from the left of the 4-digit measurement value. The third sensor 163, third from the left, receives relaxation energy of the third wavelength and may indicate the third digit from the left of the 4-digit measurement value. The fourth sensor 164, fourth from the left, receives relaxation energy of the fourth wavelength and may represent the fourth digit from the left of the four-digit measurement value.

When relaxation energy that has passed through the multi-filter is projected to the first sensor 161, 1 may be output from the first sensor 161. when relaxation energy is not projected to the second sensor 162, 0 may be output from the second sensor 162. When relaxation energy is projected to the third sensor 163, 1 may be output from the third sensor 163. When relaxation energy is not projected to the fourth sensor 164, 0 may be output from the fourth sensor 164. In reality, relaxation energy may be projected to each sensor even if there is a difference in intensity value. However, when the intensity value satisfies the set value, 1 may be output from the sensor. Likewise, when the corresponding intensity value does not satisfy the set value, 0 may be output from the sensor.

According to this, the output value may be 0101. The detector 100 may search for 0101 in the lookup table obtained through the preliminary experiment and extract information about a virus matching 0101. This may mean that the virus was detected in the specimen 10.

The relaxation energy of a virus generated by the detection electrode 170 may have light energy of different wavelengths depending on the type of virus. According to this embodiment, the relaxation energy for each wavelength of different types of viruses may be detected. Additionally, the presence or absence of a virus and the type thereof may be determined using the wavelength of the detected relaxation energy and the lookup table.

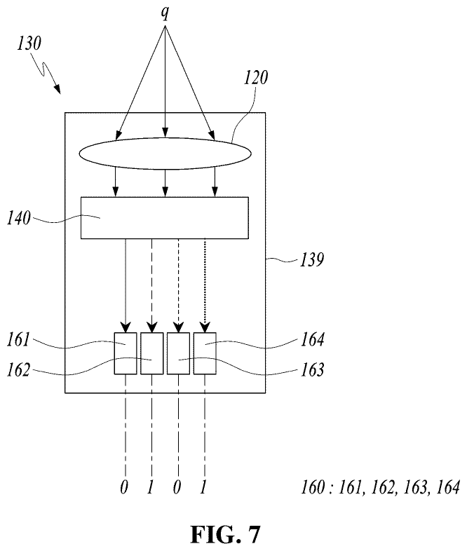

FIG. 7 is a schematic view showing a detector 100 according to another embodiment of the present disclosure.

A multi-filter of FIG. 7 may include a diffraction grating that outputs light toward different directions depending on the wavelength when a single relaxation energy is input.

At this time, sensors 160 may be respectively provided at locations where each light output from the diffraction grating arrives. The virus detection algorithm using the output value of each sensor 160 may be the same as the description regarding FIG. 6.

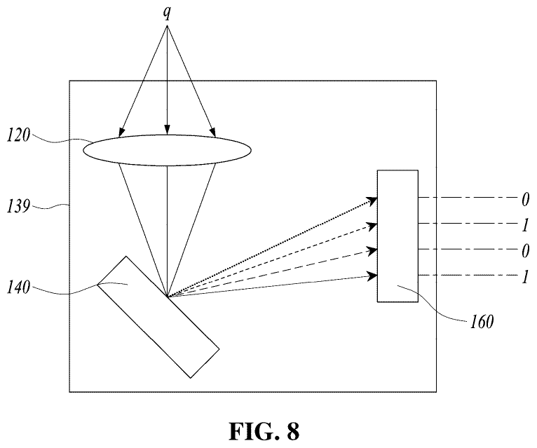

FIG. 8 is a schematic view showing a detector 100 according to still another embodiment of the present disclosure.

A multi-filter of FIG. 8 may include a diffraction grating that outputs light toward different directions depending on the wavelength when a single relaxation energy is input.

A multi-sensor of FIG. 8 may include a linear image sensor.

The linear image sensor may electrically output an output value of a set number of bits (number of digits in binary system) for each input position.

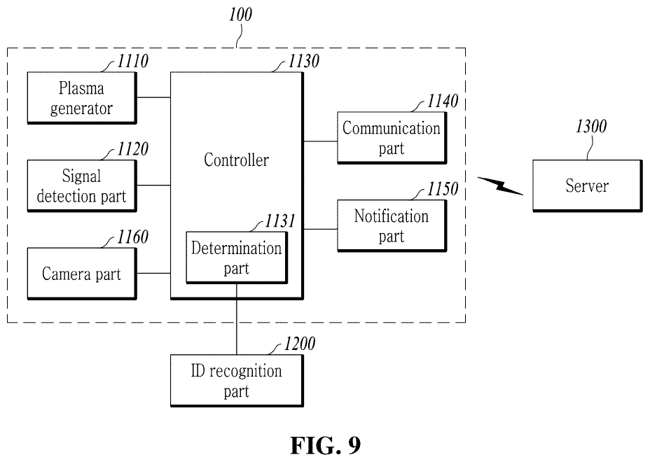

FIG. 9 is a block view showing the detector 100 according to an embodiment of the present disclosure. The detector 100 may include a plasma generator 1110, a signal detection part 1120, a controller 1130, a determination part 1131, a communication part 1140, a notification part 1150, and a camera part 1160.

The plasma generator 1110 may create a spark by supplying a voltage of 2 to 24 kV using the detection electrode 170 made of tungsten. A virus contained in the specimen 10 is decomposed by the spark, and light may be emitted as a result. The detection electrode made of tungsten may be provided in 1 to 4 pairs, but are not limited thereto. At this time, one pair may consist of two or three.

The plasma generator 1110 is configured in the form of an electric flyswatter to form a spark, so that both sides thereof have negative electricity, and three electrodes that have positive electricity may be implemented in the center thereof to form plasma.

The signal detection part 1120 may measure the spectrum of light L emitted through the plasma generator 1110.

The signal detection part 1120 may include the lens 120 that collects the emitted light L, the detection filter 140 that passes a preset wavelength of light passing through the lens 120, and the sensor 160 that detects the light passing through the detection filter 140. The detection filter 140 may include a bandpass filter, and the sensor 160 may include a photodiode.

In this case, the sensor may include 1 to 8 photodiodes, but is not limited thereto.

The controller 1130 may control the starting point of operation of the signal detection part 1120 so that the signal detection part 1120 is driven after a preset time.

The determination part 1131 may determine whether there is a virus and the type of virus by combining the data measured by the spectrum, and measure the virus concentration. In this case, to improve the accuracy of virus identification results, an artificial intelligence (AI) model that analyzes the line spectrum according to virus type may be applied. The artificial intelligence model may be deep learned based on models such as a convolutional neural network (CNN) and a recurrent neural network (RNN).

The communication part 1140 may transmit the virus determination result and the face image recognized by means of a camera to a server 1300. In this case, the server 1300 may constitute a control system managed by an administrator. The server is installed in the central control room or central control room, and may transmit data provided from the communication part 1140 to the administrator's terminal (equipped with a web app or mobile app).

The notification part 1150 may display the virus detection results through a speaker or display.

The camera part 1160 may recognize the user's face.

In some cases, an ID recognition part 1200 may be added.

The controller 1130 may match the user 20 and the virus detection result using the face recognized by the camera part 1160 or the ID card recognized by the ID recognition part 1200.

In addition, the controller 1130 may distinguish between the current user 20 and the next user 20 using the recognized face or ID card. The controller 1130 may determine the disinfection time of the sterilizer 180 using the distinction result.

An insertion hole into which a funnel-shaped straw is inserted may be formed in the center of the cover 210. The straw may directly receive input from the user's exhalation. Alternatively, the user's exhaled breath may be input from a balloon inflated by the user's breath. In this case, a foreign substance filter may be provided inside the straw to remove impurities such as dust. When the straw is provided, the insertion hole may replace the guide hole 207 of the cover 210.

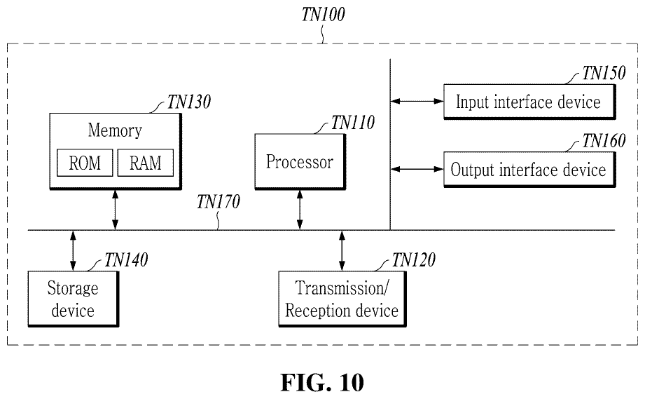

FIG. 10 is a diagram showing a computing device according to an embodiment of the present disclosure. The computing device TN100 of FIG. 10 may be an apparatus (e.g. the detection apparatus 1000, etc.) described herein.

In the embodiment of FIG. 10, the computing device TN100 may include at least one processor TN110, a transceiver device (transmission/reception device) TN120, and a memory TN130. The computing device TN100 may further include a storage device TN140, an input interface device TN150, an output interface device TN160, etc. Components included in the computing device TN100 may be connected by a bus TN170 to communicate with each other.

The processor TN110 may execute a program command stored in at least one of the memory TN130 and the storage device TN140. The processor TN110 may refer to a central processing unit (CPU), a graphics processing unit (GPU), or a dedicated processor in which methods according to an embodiment of the present disclosure are performed. The processor TN110 may be configured to implement a procedure, a function, and a method described in connection with the embodiment of the present disclosure. The processor TN110 may control each component of the computing device TN100.

Each of the memory TN130 and the storage device TN140 may store various pieces of information related to the operation of the processor TN110. Each of the memory TN130 and the storage device TN140 may include at least one of a volatile storage medium and a non-volatile storage medium. For example, the memory TN130 may include at least one of a read only memory (ROM) and a random access memory (RAM).

The transceiver device TN120 may transmit or receive a wired signal or a wireless signal. The transceiver device TN120 may be connected to a network to perform communication.

Although the embodiments of the present disclosure have been described above in the detail, the scope of rights of the present disclosure is not limited thereto, and various modifications and improvements made by those skilled in the art using the basic concept of the present disclosure defined in the following claims also fall within the scope of rights of the present disclosure.

What is claimed is:

1. A detection apparatus comprising:
a detector configured to detect a virus in a specimen containing an exhaled breath of a user.

2. The apparatus of claim 1, wherein a collection container is provided to receive the specimen, and the detector is installed in the collection container and detects a virus in the specimen entered into the collection container.

3. The apparatus of claim 2, wherein when a force of a user's exhalation is defined as a first force, a cover that receives the specimen moving by the first force is provided at an entrance of the collection container,
an input flow path connected to the detector is formed in a center of the cover, and
the specimen entered into the cover moves along the input flow path and is delivered to the detector.

4. The apparatus of claim 2, wherein when the specimen is input into the collection container along a first direction, a cover installed at an entrance of the collection container is provided,
a guide hole is provided in a center of the cover,
the guide hole has an inner diameter that decreases along the first direction,
an inspection box that is installed inside the collection container and has a receiving space for accommodating the specimen is provided,
the inspection box is connected directly to an end of the guide hole, or is connected to the end of the guide hole through a guide pipe, and
the detector detects a virus in the specimen contained in the inspection box.

5. The apparatus of claim 4, wherein a foreign substance filter is provided at the end of the guide hole to be detachable and to filter out foreign substances, and
a suction fan is installed in the collection container and arranged to face the foreign substance filter with the inspection box in between,
wherein the suction fan is provided to suck gas in the receiving space.

6. The apparatus of claim 2, wherein when the user's exhaled breath moves along a first direction, the collection container is provided to extend along the first direction,
an inspection box is provided on a first side in the collection container in the first direction,
a receiving space in which the specimen is received is formed in the inspection box,
the detector detects a virus in the specimen contained in the inspection box,
a driving unit for driving the detector is provided on a second side in the collection container,
an exhaust flow path extending along a second direction inclined to the first direction is formed in a center of the collection container in the first direction,
an output hole for the exhaust flow path is formed on a bottom of the collection container, and
the exhaust flow path is connected to an output end of the inspection box.

7. The apparatus of claim 1, wherein the detector detects relaxation energy of the specimen decomposed by at least one of thermal energy, light energy, and electrical energy, and
the detector detects the virus using a wavelength or an intensity value of the detected relaxation energy, or detects the virus using a ratio between intensity values by wavelength of the detected relaxation energy of multiple wavelengths.

8. The apparatus of claim 1, wherein an inspection box is provided with a receiving space for accommodating the specimen,
a collector is provided that collects the specimen introduced into the receiving space to a set position in the receiving space,
an optical device is provided that focuses on the set position, and
the detector detects the virus through analysis of energy passing through the optical device.

9. The apparatus of claim 1, wherein an inspection box is provided with a receiving space for accommodating the specimen,
a discharge electrode is provided at a first position in the receiving space,
a ground electrode is provided at a second position in the receiving space,
a power device is provided to apply a high voltage between the discharge electrode and the ground electrode,
an optical device is provided that focuses on a set position closer to the second position than the first position on a virtual straight line connecting the first position and the second position, and
the detector detects the virus through analysis of energy passing through the optical device.

10. The apparatus of claim 9, wherein a detection electrode is provided at the set position,
the detection electrode creates a spark that produces plasma targeting the specimen,
the detector detects relaxation energy of the specimen decomposed by the spark, and
the detector detects the virus using a wavelength of the detected relaxation energy.

11. The apparatus of claim 1, wherein an inspection box is provided with a receiving space for accommodating the specimen,
a sensor is provided to detect relaxation energy in the receiving space by wavelength, and
the detector detects the virus by using a wavelength and an intensity value at which the relaxation energy is detected, or detects the virus by using a ratio between intensity values by wavelength of the detected relaxation energy of multiple wavelengths.

12. The apparatus of claim 1, wherein an inspection box is provided with a receiving space for accommodating the specimen,
the inspection box is formed in a polyhedral shape,
a first optical device installed on a first surface of a plurality of surfaces constituting the inspection box is provided,
a second optical device installed on a second surface that is distinct from the first surface among the plurality of surfaces constituting the inspection box is provided,
when a normal line of the first surface is defined as a first normal line and a normal line of the second surface is defined as a second normal line, the first surface and the second surface are formed so that the first normal line and the second normal line intersect at a set position,
the first surface and the second surface have a same distance to the set position,
the first optical device is provided with a first detection filter that passes only energy of a first wavelength, whereas the second optical device is provided with a second detection filter that passes only energy of the second wavelength,
a first sensor is provided to sense energy that has passed through the first detection filter,
a second sensor is provided to sense energy passing through the second detection filter, and
the detector detects the virus using an output value of the first sensor and an output value of the second sensor.

13. The apparatus of claim 1, wherein an inspection box is provided with a receiving space for accommodating the specimen,
a detection electrode is provided in the receiving space,
the detection electrode creates a spark that produces plasma targeting the specimen,
the detector detects relaxation energy of the specimen decomposed by the spark, and
the detector detects the virus using a wavelength or an intensity value of the detected relaxation energy, or detects the virus using a ratio between intensity values by wavelength of the detected relaxation energy of multiple wavelengths.

14. The apparatus of claim 13, wherein a sterilizer is provided to disinfect the receiving space when a virus detection process of the detector targeting a specific user is completed,
the sterilizer outputs disinfected water containing an oxygen compound toward the detection electrode,
the detection electrode electrolyzes the disinfected water to generate oxygen, and
the receiving space is disinfected by the oxygen generated in the receiving space.

15. The apparatus of claim 1, wherein an inspection box is provided with a receiving space for accommodating the specimen,
a detection electrode is provided in the receiving space,
the detection electrode creates a spark that produces plasma targeting the specimen,
an optical tube connected to the inspection box is provided,
the optical tube is equipped with a lens, a multi-filter, and a multi-sensor,
the lens creates an optical path through which inspection light corresponding to relaxation energy formed by the plasma propagates,
the multi-filter divides the inspection light by wavelength so that when the inspection light is input, the inspection light is output toward different locations for each wavelength,
the multi-sensor receives the inspection light output from the multi-filter at different locations,
the multi-sensor outputs information about a location where the inspection light is received,
the detector extracts a combination of information about a location where the inspection light is received from a lookup table obtained through experiment, and
the detector outputs information on a virus matching the extracted combination.
